(19) United States
(12) Patent Application Publication
(10) Pub. No.: US 2024/0181473 A1
(43) Pub. Date: Jun. 6, 2024

(54) DISPENSER

(71) Applicant: WYPE LTD., London (GB)

(72) Inventors: Eli Khrapko, London (GB); Giorgia Granata, London (GB); Jo Barnard, London (GB); Lea Berger, London (GB); Juliette Bouilly, London (GB); Harry Mason, London (GB); Henry Zhou, London (GB)

(21) Appl. No.: 18/285,834

(22) PCT Filed: Mar. 17, 2022
(86) PCT No.: PCT/GB2022/050676

§ 371 Date: Oct. 5, 2023

(30) Foreign Application Priority Data
Apr. 9, 2021 (GB) 2105066.1

(51) Int. Cl. B05B 1/14; B05B 11/00; B05B 11/10

(52) U.S. Cl. CPC B05B 1/14; B05B 11/0032; B05B 11/1002; B05B 11/1047

(57) ABSTRACT

An applicator (1) for applying a gel to a sheet of paper, having a pump (5), an actuator (6), and an application surface (3) having a plurality of apertures (4). When the pump is fed with gel, actuation of the actuator causes the pump to move gel through the apertures so that gel is dispensed simultaneously through each of the apertures. The applicator can be used to create a bio-degradable wet-wipe.

Wet wipes are a popular convenience item used for personal hygiene and household cleaning. They comprise a piece of plastic or cloth that is impregnated with a fluid such as a disinfectant. Most wet wipes are made from nonwoven fabrics such as polyester or propylene.

Wet wipes cause environmental problems because they are generally not biodegradable. They can therefore clog up sewer systems and persist in the environment.

An alternative to wet wipes has recently been made available. This involves applying a specially formulated gel to a regular piece of toilet paper. This provides the benefits of a wet wipe, with the environmental benefits of standard toilet paper. This product is sold as a gel in the UK by Wype Limited, of 86-90 Paul Street, London, England, EC2A 4NE.

It is however difficult to apply a gel evenly to a sheet of paper, in the correct amounts and with the correct coverage to provide the intended wet wipe properties.

It is amongst the objects of the present invention to address one or more of these problems.

In a first aspect the invention provides an applicator for applying a gel to a sheet of paper, comprising;
a pump,
an actuator, and
an application surface having a plurality of apertures.

The plurality of apertures allow the gel to be easily distributed over the area of a regular square of toilet paper.

Preferably, when the pump is fed with gel, actuation of the actuator causes the pump to move gel through the apertures so that gel is dispensed simultaneously through each of the apertures.

Preferably the apertures are sized so that in use equal volumes of gel are dispensed through each of the apertures. This helps to provide an even distribution of gel. Preferably the apertures are circular.

Preferably the applicator is provided with between 3-9 and, more preferably 6, apertures. This provides an optimum distribution for the gel. Preferably the apertures are arranged at regular intervals around an outer edge of the application surface of the applicator.

Preferably the apertures are arranged symmetrically on the application surface of the applicator. In some embodiments the application surface is generally planar. The applicator may be provided with any practical number of apertures, for example, 10-50, 10-100, or more. The apertures may be nozzles. The apertures may be arranged in any pattern on the application surface of the applicator to obtain the optimal distribution of gel.

Preferably the application surface is circular.

Preferably the applicator further comprises a gel conduit between the pump and the apertures which comprises a splitter section for splitting a flow of gel into equal parts and directing those equal parts to corresponding apertures.

This provides a convenient way of splitting the flow from the pump.

Preferably the application surface is concave.

The largest distance between apertures is preferably between 25-35 mm and is more preferably 28-30 mm, or 28 mm. These distances may for example apply to apertures which are located on opposite sides of the application surface of the applicator. Distances between adjacent apertures may be smaller than the distances stated above. These distances may correspond to the diameter of the application surface.

The applicator is preferably elongate and the actuator is provided on a side of the applicator. This allows one handed operation of the applicator, whilst a user holds the piece of toilet paper to which the gel is being applied, in the other hand. Preferably the actuator is actuatable in a direction which is perpendicular to an axis of elongation of the applicator. Preferably the applicator is generally cylindrical.

Preferably the applicator further comprises a reclosable lid, wherein a tab on the lid covers the actuator when the lid is closed. This prevents the actuator (which may be a button) from being actuated unintentionally when the lid of the applicator is closed. The lid preferably covers the application surface when the lid is closed.

The applicator is preferably provided with an engaging means for removably engaging the applicator with a reservoir for holding gel such that, when so engaged, the pump of the applicator draws gel from the reservoir when the actuator is actuated. The engaging means may be a screw thread which engages with a corresponding thread on the neck of a reservoir. The reservoir may be a re-fillable canister, optionally made of aluminium. The engaging means may be a screw thread or a snap fit arrangement.

The applicator may be provided with a collar between outer walls of the applicator and the pump. The collar may be arranged to provide a robust attachment to a reservoir, optionally by reducing the flex in sidewalls of the applicator. The collar may also act as a seat for the actuator to provide a robust base for actuation.

The splitter section may be formed by a plate. The splitter section may have a single-entry aperture which is fluidly connected to an exit of the pump. The entry aperture allows gel exiting the pump to enter a hollowed-out volume in the plate which forms two or more, preferably 6, channels. In use the plate is located under the application surface of the applicator so that the underside of the application surface forms a top surface of the channels. The apertures in the application surface are aligned so that each aperture forms an exit for gel which is being forced through a corresponding channel. The channels have equal volumes. This arrangement requires no moving parts except for the actuator and the pump to split the gel into equal portions. It is also unlikely to become blocked.

The splitter section may be provided with an alignment means (for example a notch) to assist in the assembly of the splitter section so that the splitter section is correctly aligned with apertures on the application surface. The splitter section may be a curved plate. The curve of the splitter section may correspond to the underside of a curved application surface of the applicator (in which the apertures are located).

The applicator may be provided with a grab handle, which may optionally be attached to a collar of the applicator.

In a further aspect the invention provides a dispenser comprising an applicator as described herein and a reservoir for accommodating a gel.

The reservoir may be removably engageable with the applicator.

The apertures and pump may be arranged to dispense between 0.1-1 ml, preferably 0.25-0.35 ml and more preferably 0.3 ml or 0.5 ml, of gel per actuation of the actuator.

The reservoir preferably contains a gel. Preferably the gel comprises the extract of an Aloe plant, and optionally water. Preferably the gel comprises Aloe barbadensis leaf juice.

Preferably the gel comprises one or more of Alcohol Denat, Glycerin, Propanediol, Tocopherol (Vitamin E), Decyl Glucoside, Carbomer, Phenoxyethanol, Sodium Hydroxide and Ethylhexylglycerin.

Preferably the gel comprises one or more of Glycerin, Xanthan gum, decyl glucoside, propanediol, benzyl alcohol, citric acid, tocopherol (vitamin E), dehydroacetic acid, Chamomilla recutita (chamomile) flower extract, Cocos nucifera (coconut) fruit extract, Bambusa vulgaris (bamboo) leaf extract, sodium benzoate and potassium sorbate.

In a further aspect the invention provides a method of dispensing a gel from a dispenser as described herein, onto toilet paper, by actuating the actuator of the applicator and wiping the toilet paper on the application surface of the applicator.

A preferred embodiment of the invention will now be described with reference to the drawings in which;

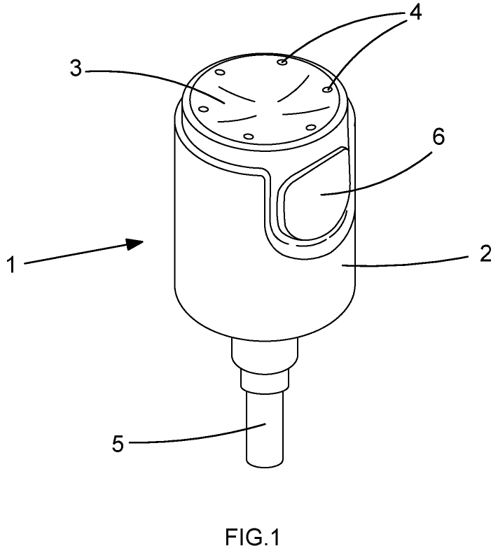

FIG. 1 shows a perspective view of an applicator according to an embodiment of the present invention.

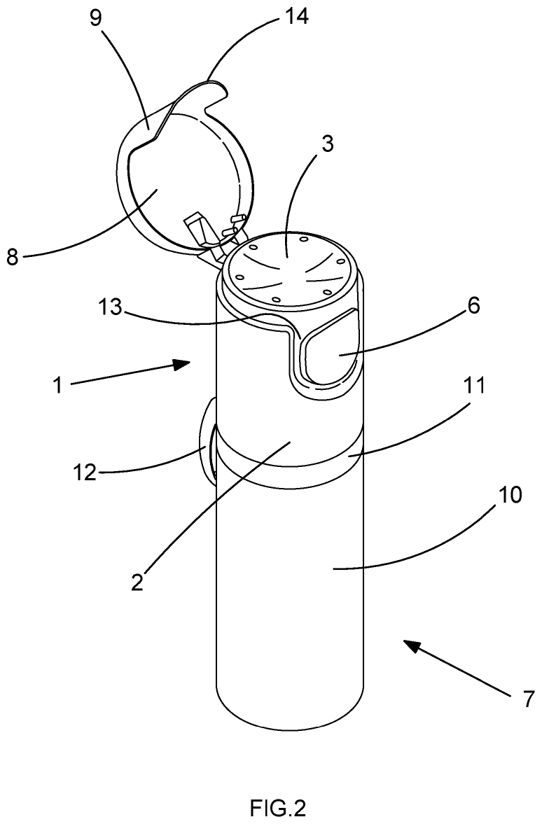

FIG. 2 shows a perspective view of a dispenser according to an embodiment of the invention, which includes an applicator according to the invention.

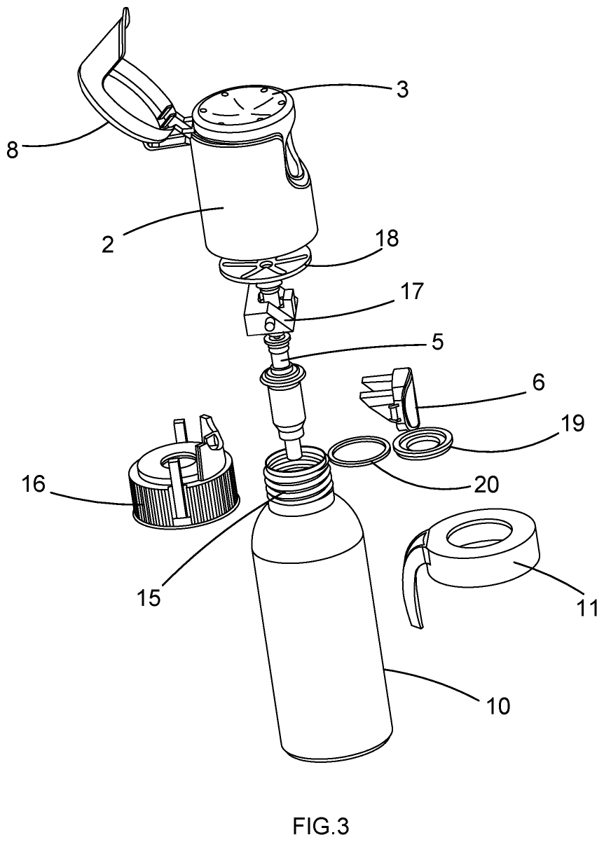

FIG. 3 shows an exploded view of the dispenser shown in FIG. 2.

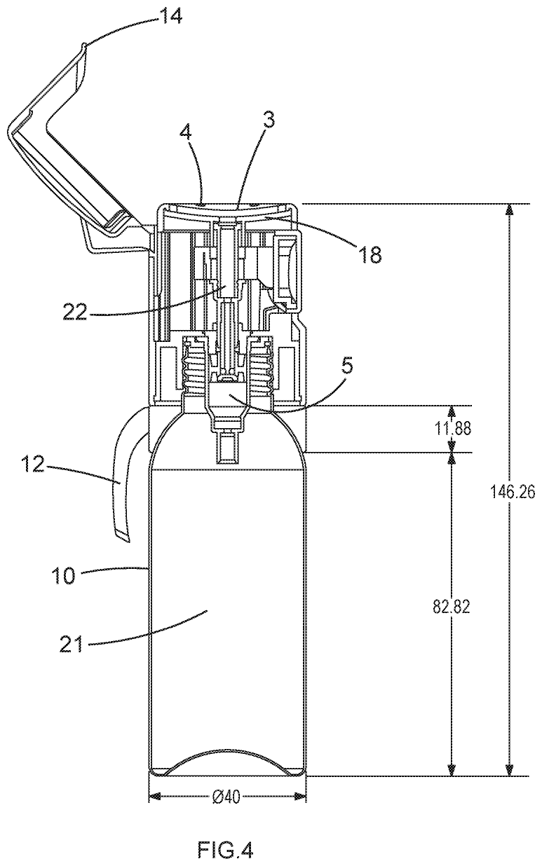

FIG. 4 shows a non-exploded, cross sectional view of the dispenser shown in FIG. 3.

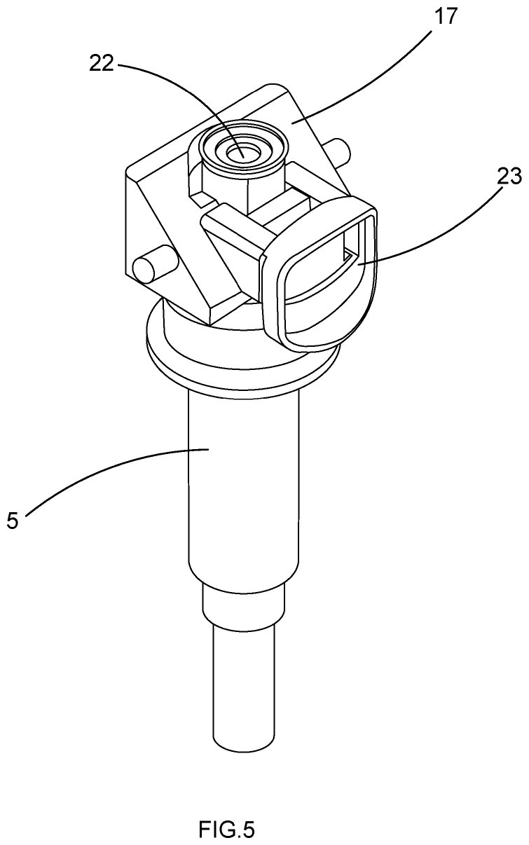

FIG. 5 shows a partial view of components of the applicator shown in FIG. 1.

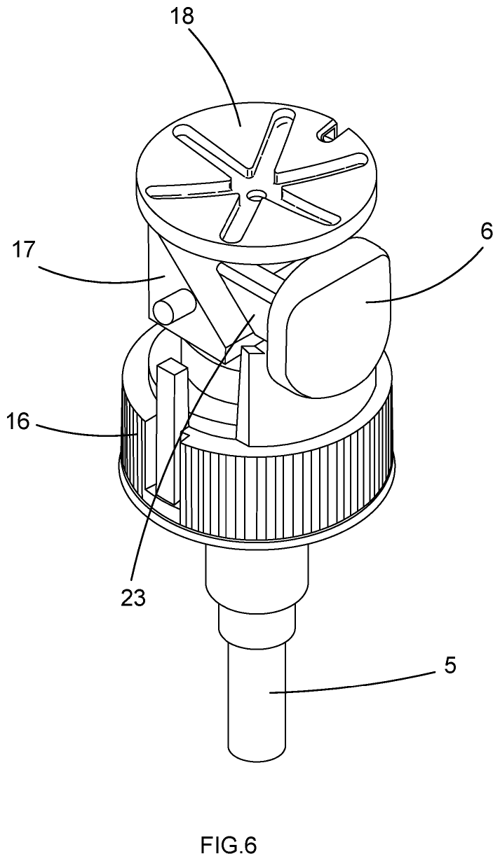

FIG. 6 shows a second partial view of components of the applicator shown in FIG. 1.

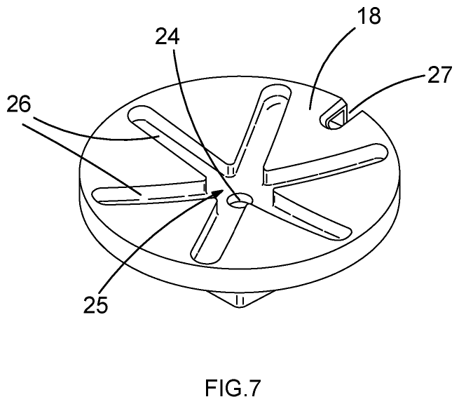

FIG. 7 shows a perspective view of a splitter section which may be used in applicators according to the invention, such as that of FIG. 1.

FIG. 1 shows a perspective view of an applicator 1 according to the invention. The applicator comprises an elongate body having an outer wall 2 which has a generally cylindrical cross section when viewed from above. The applicator has a generally circular top application surface 3. Around the perimeter of the top surface, six apertures 4 are located. The apertures are regularly spaced. This arrangement provides the maximum possible symmetry.

A pump 5 is centrally located along the cylindrical axis of the applicator. The lower end of the pump comprises a tube which hangs below the outer wall 2 of the applicator and which is adapted to suck up a gel as the pump is actuated. An actuation button 6 is located in a recess on one side of the elongate body. The actuation button is linked to the pump such that when the button is pressed by a user, a pre-determined amount (0.45-0.55 ml) of gel is sucked into the pump. Gel is therefore urged by the pump through the apertures 4. The apertures all have the same cross-sectional (circular) area.

In use, gel is forced out of the apertures as the button is actuated. A discrete and uniform amount of gel is forced out of each aperture. In this embodiment around 0.075 ml-0.092 ml of gel will exit each aperture by a single actuation of the actuator button. The applicator is then turned over and the gel is wiped onto a piece of toilet paper, to form the wet wipe substitute. The application surface is slightly convex to assist in the wiping and spreading of the gel on the toilet paper.

FIG. 2 shows a dispenser 7 comprising an applicator 1 according to FIG. 1, and a reservoir 10.

The applicator is provided with a reclosable lid 8, which is in its open position. When the lid is closed it covers the application surface 3. The lid has a tab 9 which extends downwardly from a top surface of the lid. When the lid is closed, the tab 9 covers the actuator button 6. When the lid is closed, the tab 9 sits flush to the outer wall 2 of the applicator since the tab is accommodated in the recess 13 in the sidewall of the applicator (which also accommodates the actuator button). The tab 9 is provided with a flange 14 at its lower end. The flange 14 sits proud of the sidewall 2 when the lid is in its closed position. This allows a user to hold the dispenser in one hand, flick open the lid with their thumb and then press the actuation button, without needing to use their other hand.

The applicator is connected by a screw thread to the top of the reservoir 10 such that the pump of the applicator hangs down into the volume defined by the reservoir. Between the applicator 1 and the reservoir 10 sits an annular collar 11. A grab handle 12 is attached to the collar on an opposite side of the reservoir to the actuation button 6. The grab handle is elasticated.

The reservoir contains a gel. Two possible formulations for the gel are:
1) Aqua (Water), Aloe barbadensis Leaf Juice, Alcohol Denat, Glycerin, Propanediol, Tocopherol (Vitamin E), Decyl Glucoside, Carbomer, Phenoxyethanol, Sodium Hydroxide and Ethylhexylglycerin.
2) Aqua (Water), Aloe barbadensis Leaf Juice, Glycerin, Xanthan gum, decyl glucoside, propanediol, benzyl alcohol, citric acid, tocopherol (vitamin E), dehydroacetic acid, Chamomilla recutita (chamomile) flower extract, Cocos nucifera (coconut) fruit extract, Bambusa vulgaris (bamboo) leaf extract, sodium benzoate and potassium sorbate.

FIG. 3 shows an exploded view of the dispenser shown in FIG. 2. The screw thread 15 on the neck of the reservoir for joining the applicator to the reservoir is shown. An annular collar 16 sits between the outer walls 2 of the applicator and the neck of the reservoir to provide robustness to the outer walls 2 of the applicator. Behind the actuator button sits a ramp member 17. Ramps on the rear of the actuator button 6 interact with ramps on the ramp member 17 to convert lateral movement of a user pushing the actuator button, into a vertical movement. The ramp member is connected to the pump so that the vertical movement actuates the pump 5.

A splitter plate 18 is connected to the top of a gel conduit which extends through the pump and ramp member 17. The splitter plate sits inside the applicator in its assembled configuration, just below the application surface 3 of the applicator. The splitter plate is shown in more detail in FIG. 7.

Two O-rings 19 and 20 sit between the pump 5 and the collar 16 to provide a sealing connection between the applicator 1 and the reservoir 10.

FIG. 4 shows a cross-sectional, non-exploded view of the dispenser shown in FIG. 3. The gel 21 is shown inside the reservoir 10. Upon actuation of the pump 5, gel is sucked up through the pump, through the fluid conduit 22, into the splitter plate 18, and through the apertures 4. In this view the concave nature of the application surface 3 and the splitter plate 18 is visible. This concave surface is formed by a single, smooth curve.

FIG. 5 shows a partial internal view of components of the applicator shown in FIG. 1. The fluid conduit 22 is shown at the upper end of the pump 5. The ramp member 17 is also shown engaged with the pump to convert the horizontal action of the actuation button into a vertical motion suitable for driving the pump. The housing 23 of the actuator button is shown. This provides a base for the actuation button 6 (not shown in this figure) and communicates the movement of the actuation button to the ramp member 17.

FIG. 6 shows a second partial internal view of components of the applicator shown in FIG. 1. The components are identical to those in FIG. 5, except for the fact that the actuation button 6 is shown in its position in the housing 23 and the splitter plate 18 is attached to the top of the fluid conduit 22. The collar 16 is also shown.

FIG. 7 shows a perspective view of a splitter section. The splitter section comprises a plate 18 which may be used in applicators according to the invention, such as that of FIG. 1. The splitter plate has a single circular entry aperture 24 to allow gel to exit the fluid conduit 22 and enter into the void 25 defined by the splitter plate. The void has a central hub and six arms 26 which extend from the central hub. The splitter plate, when in situ, sits beneath the concave application surface 3 of the applicator so that the underside of the application surface encloses the void 25. Gel fills up the void as it is pumped from the reservoir. The splitter plate is fitted to the applicator so that the apertures 4 in the application surface 3 correspond to each arm of the void 25. Once the void is full of gel, each further actuation of the pump will force a pre-determined amount of gel through each of the apertures 4. Since the arms are of equal volume and the void 25 is symmetrical, an identical amount of gel will be forced through each aperture. The void has 3 axes of symmetry but may have more axes of symmetry in other embodiments. This allows a user to spread the gel evenly and consistently onto a piece of toilet paper, whichever way the dispenser is held. The splitter plate 18 is provided with an alignment means, for example the notch 27. This alignment means can be aligned with a corresponding alignment means on the applicator to ensure that when the applicator is assembled, the arms of the void 25 match up with the apertures 4.

1. A dispenser comprising:
a reservoir which contains a gel comprising water, wherein the gel is suitable for application to a sheet of paper; and
an applicator configured to apply the gel to the sheet of paper, wherein the applicator comprises:
a pump,
an actuator, and
an application surface which is oriented perpendicular to an axis of elongation of the applicator and has a plurality of apertures;
wherein, when the pump is fed with the gel, actuation of the actuator causes the pump to move the gel through the apertures so that the gel is dispensed simultaneously through each of the apertures;
wherein the reservoir is removably engageable with the applicator;
wherein the applicator comprises an engaging element configured to removably engage the applicator with the reservoir such that, when engaged, the pump of the applicator draws the gel from the reservoir when the actuator is actuated; and
wherein the applicator is elongate and the actuator is provided on a side of the applicator and is actuatable in a direction which is perpendicular to an axis of elongation of the applicator.

2. The dispenser according to claim 1 wherein the apertures are sized so that in use equal volumes of gel are dispensed through each of the apertures.

3. The dispenser according to claim 1 wherein the applicator comprises between 3-9 apertures or 6 apertures.

4. The dispenser according to claim 1 wherein the apertures are arranged symmetrically on the application surface of the applicator.

5. The dispenser according to claim 1 wherein the application surface is circular.

6. The dispenser according to claim 1 further comprising a gel conduit between the pump and the apertures which comprises a splitter section configured to split a flow of gel into equal parts and to direct those equal parts to corresponding apertures.

7. The dispenser according to claim 1 wherein the application surface is concave.

8. The dispenser according to claim 1 wherein the largest distance between the apertures is between 25-35 mm or 28 mm.

9. (canceled)

10. The dispenser according to claim 1 further comprising a reclosable lid, wherein a tab on the lid covers the actuator when the lid is closed.

11-13. (canceled)

14. The dispenser according to claim 1 wherein the apertures and pump are arranged to dispense between 0.1-1 ml or 0.25-0.35 ml of gel per actuation of the actuator.

15. The dispenser according to claim 1 wherein the gel comprises an extract of an Aloe plant.
